(19) United States
(12) Patent Application Publication
(10) Pub. No.: US 2010/0170514 A1
(43) Pub. Date: Jul. 8, 2010

(54) AIR FILTER FOR A MASK ASSEMBLY

(75) Inventor: Marina Omer-Cooper, Carnegie (AU)

Correspondence Address: SUGHRUE MION, PLLC, 2100 PENNSYLVANIA AVENUE, N.W., SUITE 800, WASHINGTON, DC, 20037, US

(73) Assignee: Compumedics Medical Innovations Pty. Ltd., Abbotsford (AU)

(21) Appl. No.: 12/376,931

(22) PCT Filed: Jul. 24, 2007
(86) PCT No.: PCT/AU07/01016

§ 371 Date: Mar. 4, 2009

(30) Foreign Application Priority Data
Aug. 9, 2006 (AU) 2006904290

(51) Int. Cl. A61M 16/06; A62B 7/10

(52) U.S. Cl. 128/205.12; 128/205.27; 128/206.12

(57) ABSTRACT

The present invention provides a gas-delivery system for delivering breathable gas to a subject, comprising a source of pressurised gas, a mask cushion, a means for positioning the mask cushion for delivery of gas, and a filter for filtering particles from exhaled air. Preferably, the filter filters exhaled air only. The filter may engage a mask frame on any suitable surface of the frame in relation to the mask cushion.

### FIELD OF THE INVENTION

The present invention relates to masks for the provision of gases to patients, in particular, positive airway pressure masks and mask assemblies.

### BACKGROUND

There are a number of treatments for patients that require the use of a mask for the delivery of gas, including air, to a patient's nose. For example, in the treatment of sleep apnoea, in particular, obstructive sleep apnoea (OSA), air is often delivered at continuous positive airway pressure (CPAP), automatic positive airway pressure (APAP), or variable positive airway pressure (VPAP) wherein air is supplied at a pressure greater than ambient to the nose of a sleeping patient through a mask to keep the patient's airways open for effective respiration. Often air must be delivered through a mask for sustained time periods, for example, through the whole of an overnight period of sleep.

It is important for continuous therapeutic benefit to be achieved from pressurised air treatment that a mask assembly be comfortable and relatively leak-proof. A mask must maintain its position over the nose and seal around the nose or with the face of a patient to maintain positive airway pressure when required despite the movements of the patient during sleep that might break the seal. At the same time, the mask must be comfortable so that a patient achieves the therapeutic benefit of relatively unbroken sleep periods.

Various filtration systems have been developed to achieve adequate filtering of air supplied under positive pressure to the nose of a patient using CPAP and VPAP devices. These filters are designed to filter the air before it enters the patient's airways.

Treatment with pressurised gas systems may be extended, during which time a patient may suffer various infectious diseases. It is important to maintain pressurised gas treatment even during such periods. However, it is also increasingly important that the patient using the CPAP and VPAP devices in the comfort of their home or in a sleep clinic staff does not inadvertently become a source of infection for others.

What is needed is a positive airway pressure mask assembly that accommodates the ability to reduce the probability of inadvertent infection of others while maintaining normal sleeping conditions for patients.

### BRIEF DESCRIPTION OF THE DRAWINGS

FIG. 1 shows a perspective view of a mask assembly including headgear, cushion and exhalation filter.

FIG. 2 shows an exploded view of a mask assembly including headgear, cushion and exhalation filter with the filter in a first arrangement.

FIG. 3 shows an exploded view of a mask assembly including headgear, cushion and exhalation filter in a second orientation with the filter in a second arrangement.

FIG. 4 shows an exploded shows view of a mask assembly including headgear, cushion and exhalation filter in a third orientation with the filter in a third arrangement.

FIG. 5a shows a side perspective view of the passage of gas through the conduits of a mask assembly and an exhaled gas filter.

FIG. 5b shows a top view of an embodiment of an exhaled gas filter according to the invention.

### SUMMARY OF THE INVENTION

It is an object of the invention to provide a filter for a gas-delivery system that removes particles from the exhaled air of a subject. It is a further object of the invention to provide an exhalation filter that is effective but relatively easy to engage and disengage with the gas-delivery system.

In one aspect, the invention provides a gas-delivery system for delivering breathable gas to a subject, comprising a source of pressurised gas, a mask cushion, a means for positioning the mask cushion for delivery of gas, and a filter for filtering particles from exhaled air. Preferably, the filter filters exhaled air only. The filter may engage a mask frame on any suitable surface of the frame in relation to the mask cushion. In another aspect, the invention provides a mask assembly for delivering breathable gas to a subject, comprising a mask frame a mask cushion, a headgear for engaging the mask frame and mask cushion; and a filter for filtering particles from exhaled air. The filter may engage the mask frame.

### DETAILED DESCRIPTION OF THE FIGURES AND MOST PREFERRED EMBODIMENT OF THE INVENTION

As far as the author is aware, there is no gas delivery mask available for positive airway pressure treatment of patients that is can filter the air exhaled by a patient. The present invention provides a means to filter exhaled gases delivered through delivery means such as CPAP and VPAP devices. It will be understood that a reference to CPAP in this specification includes a reference to VPAP or any other positive airway pressure gas-delivery system that delivers pressurised gases through a mask to the nose of a patient. Most advantageously, the filtration means is simple to manufacture but it is small in size and easy to use.

The invention provides a mask assembly comprising members that include an exhalation filter that can be mounted on a mask collar in various associations with other mask assembly members, including mask cushion and headgear. Several embodiments of the invention are described herein. It will be understood that the scope of the invention includes embodiments not shown herein.

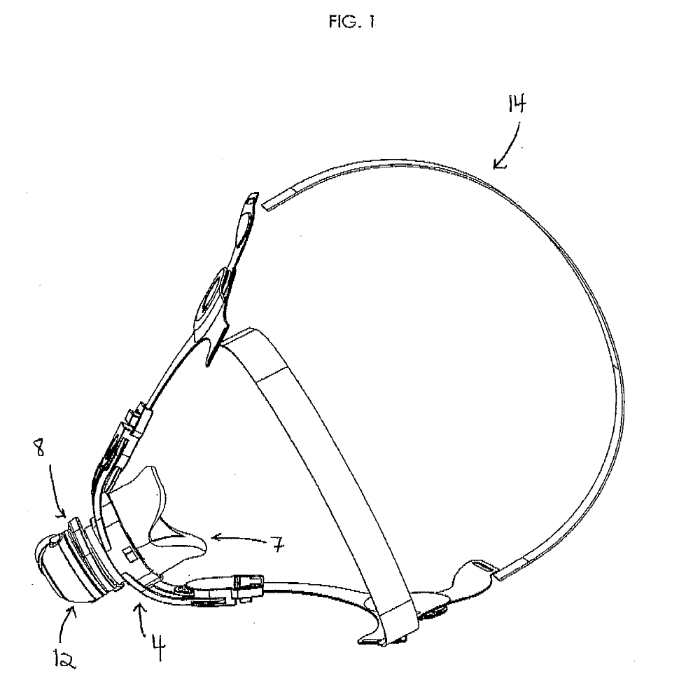

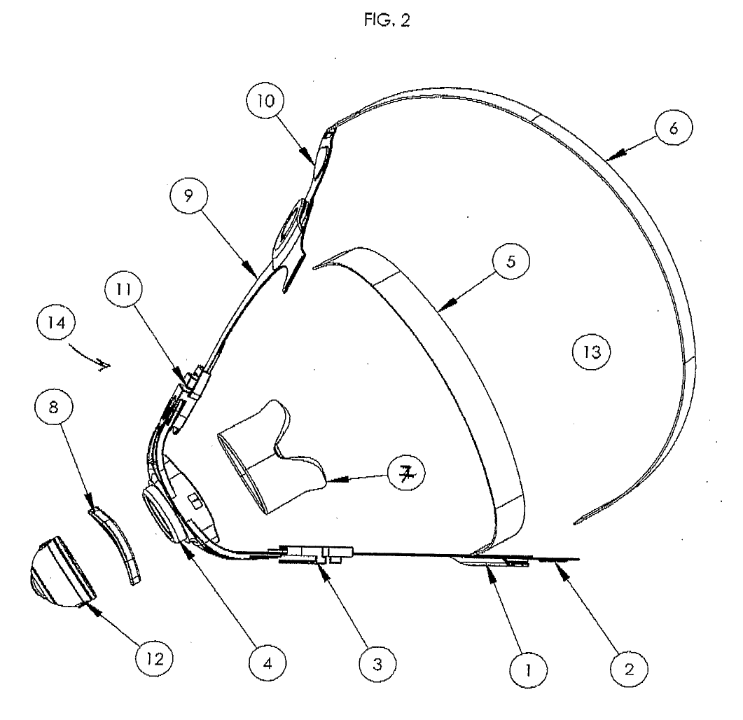

FIG. 1 shows an embodiment of a mask assembly 14 with exhalation filter 8 operatively positioned in engagement with a mask frame 4 on one side of the exhalation filter and pressurised air conduit 12 on the other side of the filter. FIGS. 2, 3, and 4 show three alternative embodiments of the invention with the exhalation filter 8 positioned differently in relation to the mask frame 4, mask cushion 7, and pressurised air conduit connector 12.

FIG. 2 shows an embodiment of the invention, being a mask assembly 14 having an exhalation filter 8 positioned and engaged with the mask frame 4 by any suitable engagement means. The engagement means for engaging the filter with the mask frame may be any of a mechanical means, such as a snap connector, VELCRO™, or chemical means such as glue, or any other engagement means that provides an effective engagement of members that resists disengagement by the movement of a patient's head during sleep but also enables quick disassembly for disposal or cleaning of the exhalation filter. In FIG. 2 the exhalation filter is positioned on the opposite of the mask frame 4 from the mask cushion 7. In this embodiment the exhalation filter 8 is also engaged with a pressurised air conduit connector 12 for delivery of gas to the mask cushion 4.

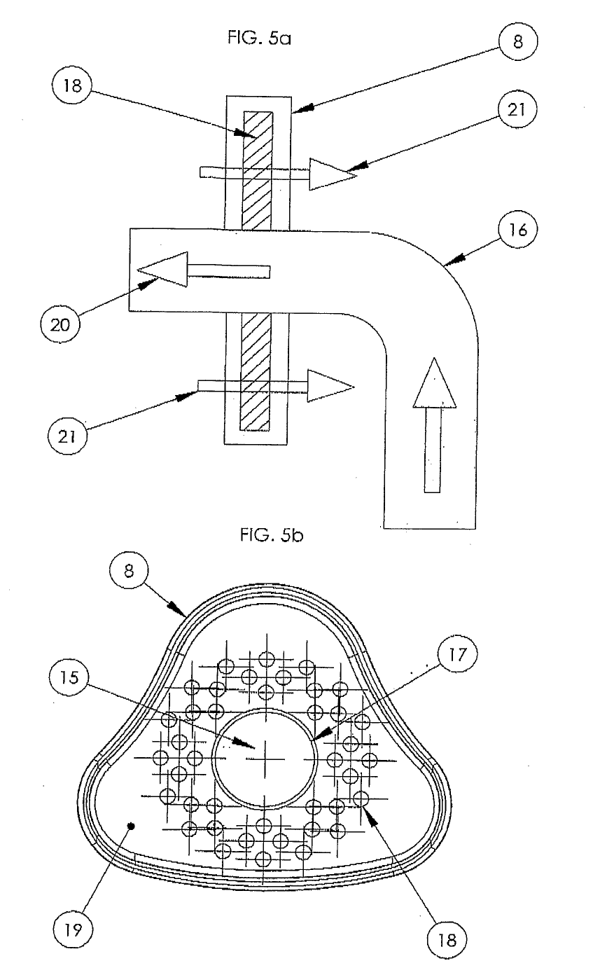

The simple design and thickness of the walls make the exhalation filter 8 relatively simple to manufacture. An embodiment of the filter 8 is shown in FIG. 5b. The perimeter 18 of the filter may take any shape that is suitable for engagement with the members, such as the mask frame, described herein. The embodiment shown in FIG. 5b shows the perimeter as being generally triangular in shape. It will be understood that the shape of the perimeter may take many alternative embodiments within the scope of the invention and is not limited to the shape shown herein. The filtration material 19 is comprised of any suitable material that allows passage of exhaled gas while filtering and removing particles such as bacteria and viral particles from the exhaled gas stream. Said filtration material is preferably a plastic or resilient, biocompatible, pliable, tear-resistant elastomeric material. In one embodiment, the filtration material 19 defines an aperture 15 through which inhalation air flows to the volume defined by the cushion 7 of the mask assembly and the face of a patient when positioned for gas delivery. Any suitable embodiment of the air filter where the inhalation air is not filtered by the exhalation filter 8 may be used.

FIG. 5a shows the flows of gases to the volume defined by the mask cushion 7 in the operation of a gas delivery apparatus embodying the invention. Pressurised air is delivered to the mask cushion through a conduit 16 in the direction shown at 20 through the connector 12 which engages the mask frame 4. The connector 12 includes an internal passageway 16 for delivering gas. The connector 12 engages engagement means15 defining the aperture of the mask frame 4. The engagement means 15 is shown in this embodiment as an elevated ridge. The aperture defined by the engagement means 15 is wider than that of the internal passageway 16 of the conduit. The filter material defines an aperture 15 having no filtering material (shown in FIG. 5b) in the portion of the conduit in communication with the aperture defined by the internal passageway 16 of the conduit so that the flow of pressurised air delivered from the gas delivery device is not impeded in any way. The aperture of the mask frame 4 includes a larger area than that of the conduit, the filtration material 18 of the filter 8 being located in the flow of exhaled gas 21. The scope of the invention includes other embodiments, as illustrated in the Figures and further embodiments, for example, including exhaust ports in the connector 12 of the conduit engaged with the mask frame 4. In each case the pathway of the pressurised gas delivered to the volume defined by the mask cushion 7 positioned on a face does not include filtration material but a filter is located in the pathway of exhaled gases.

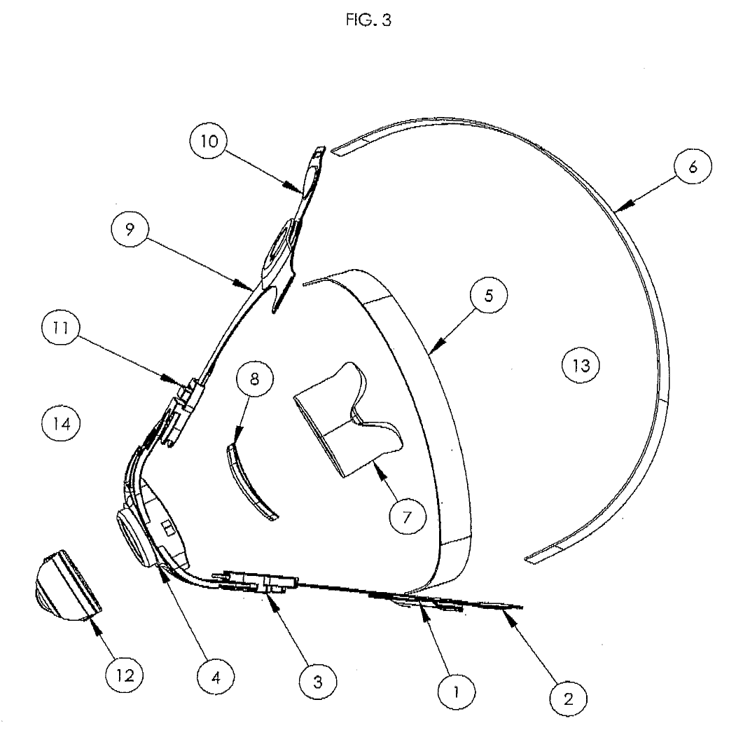

FIG. 3 shows an alternative embodiment of the invention. In this embodiment exhalation filter 8 is positioned and secured by mechanical or any other means on the inside of the mask collar 4 and engages the mask cushion 7. The quick release pivoting hose connector 12 is fitted last, securing the whole assembly in place prior to the therapeutic CPAP treatment.

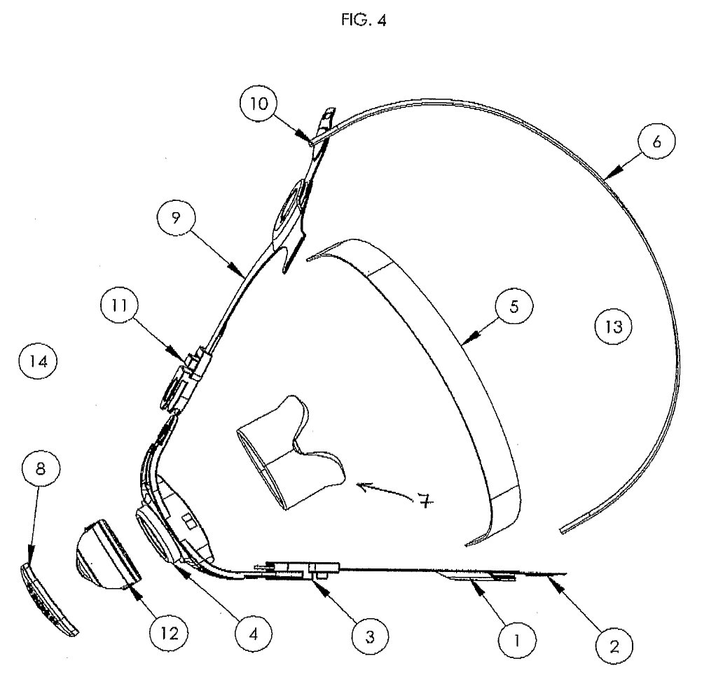

FIG. 4 shows a collar 14 assembly with the optional exhalation filter 8 according to a further embodiment of the invention. The exhalation filter positioned and secured by mechanical or any other means on the outside of the quick release pivoting hose connector 12 prior to the tarapeutic CPAP treatment.

It will be understood by those skilled in the art that the invention provides many different aspects and the objects can be achieved with embodiments of the features shown in the drawings herein and embodiments not shown in the drawings. The scope of the invention is not limited by any particular combination of features shown or described herein. The lists of examples provided herein are not exhaustive and will be apparent to a person skilled in the art in practicing the invention herein described and claimed.

1. A mask assembly for a gas-delivery system for delivering breathable gas to a subject, the mask assembly incorporating a filter for exhaled gas.

2. A gas-delivery system for delivering breathable gas to a subject, comprising:
a source of pressurised gas;
a mask cushion;
means for positioning the mask cushion for delivery of gas; and
a filter for filtering particles from exhaled gas.

3. The gas-delivery system of claim 2, wherein the filter filters exhaled gas only.

4. The gas-delivery system of claim 1, further comprising a frame for engaging the filter.

5. A mask assembly for delivering breathable gas to a subject, comprising:
a mask frame;
a mask cushion;
a headgear for engaging the mask frame and mask cushion; and
a filter for filtering particles from exhaled gas.

6. The mask assembly of claim 5 wherein the filter engages the mask frame.

7. The mask assembly of claim 5, wherein the filter filters exhaled gas only.
